(19) United States
(12) Patent Application Publication
(10) Pub. No.: US 2025/0313536 A1
(43) Pub. Date: Oct. 9, 2025

(54) PROCESS FOR RECOVERING SAFLUFENACIL FROM PROCESS STREAMS

(71) Applicant: BASF AGRO B.V., Arnhem (NL)

(72) Inventors: Gene Libey, Hannibal, MO (US); John Flanigan, Kearney, MO (US); David A. Harney, Palmyra, MO (US); David A. Cortes, Palmyra, MO (US); Petra Deckert, Bammental (DE); Keith W. Rosenkrans, Palmyra, MO (US)

(21) Appl. No.: 18/867,842

(22) PCT Filed: May 22, 2023
(86) PCT No.: PCT/EP2023/063552

§ 371 Date: Nov. 21, 2024

(30) Foreign Application Priority Data
May 30, 2022 (EP) 22176159.6

(51) Int. Cl. C07D 239/54

(52) U.S. Cl. CPC C07D 239/54

(57) ABSTRACT

A process for increasing the yield of a process for the preparation of Compound (I), in which Compound (I) is obtained in solubilized form in an organic solvent phase, crystallized and separated from the mother liquor, comprising (I) STEP I) wherein the mother liquor is extracted with a basic buffer solution, adjusting the pH between 7 and 10 and extracting Compound (I) into the aqueous phase; and STEP II) wherein Compound (I) is back-extracted from the aqueous phase by decreasing the pH to below 6 in the presence of organic solvent.

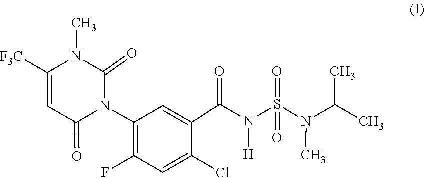 [SMILES: CC(C)N(C)S(=O)(=O)NC(=O)c1cc(-n2c(=O)cc(C(F)(F)F)n(C)c2=O)c(F)cc1Cl]

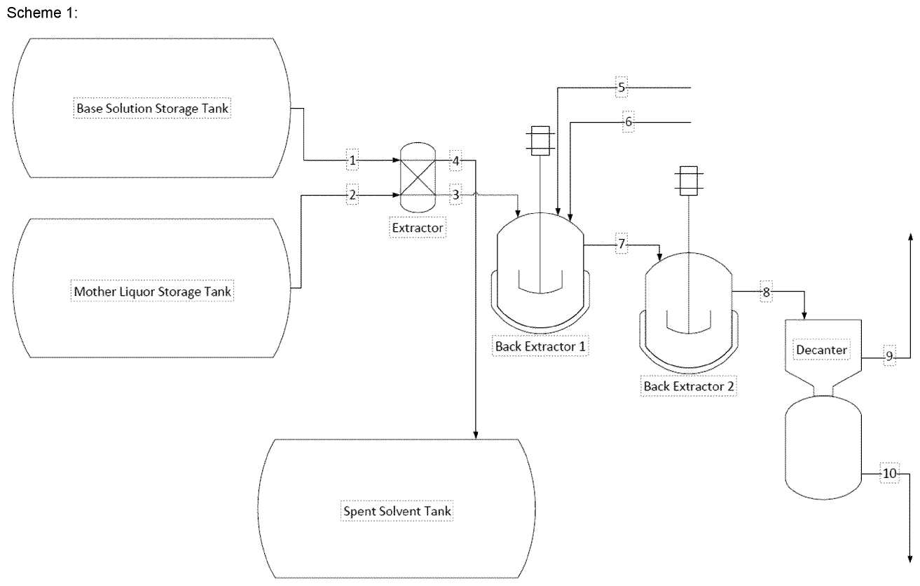

The present invention relates to a new and highly efficient process for the production of the herbicide Saflufenacil.

Saflufenacil (Compound (I)) is the common name of the compound 2-chloro-5-[3,6-dihydro-3-methyl-2,6-dioxo-4-(trifluoromethyl)-1-(2H) pyrimidinyl]-4-fluoro-N-[[methyl (1-methylethyl)-amino]sulfonyl]benzamide, which is herbicidally active, inhibiting the plant enzyme protoporphyrinogen oxidase (PPO).

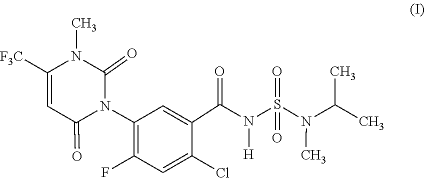 [SMILES: CC(C)N(C)S(=O)(=O)NC(=O)c1cc(-n2c(=O)cc(C(F)(F)F)n(C)c2=O)c(F)cc1Cl]

Saflufenacil has been described in WO2001/083459. Further processes for its preparation are described in WO2003/097589, WO2005/054208, WO2003/097589, WO2006/125746 and WO2008/043835. Saflufenacil is particularly useful for preplant applications and selective preemergence weed control in multiple crops, including corn and soybean. Saflufenacil as described herein includes also different forms of the compound, such as crystalline or particle forms.

In the final reaction step of the process for the preparation of Saflufenacil, the compound is obtained in solubilized form in the organic solvent phase. Subsequently, worked-up is performed according to known methods. WO2008/043835 discusses the crystallization of solubilized Saflufenacil.

However, during the work-up procedure, a part of the process yield may be lost. Up to 10% of Saflufenacil produced in the final step of the process can remain in the mother liquor after crystallization and is discarded as waste. This leads to increased raw material cost and procedurally laborious waste disposal. “Mother liquor” is the term generally used for liquids or solutions remaining after a component has been removed by a process such as crystallization and filtration.

Generally, there is a high demand to save raw materials, to reduce cost and to minimize the environmental impact of chemical processes.

Accordingly, it is an object of the present invention to provide a highly efficient process for increasing the overall yield in a process for the preparation of Saflufenacil. In addition, it is an object of the present invention to provide a highly efficient process for recovering Saflufenacil in high yield and with high purity from organic solvents. In particular, it is an object of the present invention to provide a highly efficient process for recovering the part of Compound (I), that remains solubilized in the mother liquor after crystallization of the product from the reaction mixture.

These and further objectives are achieved by the process described below.

A variety of different synthesis routes is known to the skilled person to prepare Compound (I), Saflufenacil. Generally, Compound (I) is obtained in solubilized form in the organic solvent phase.

Accordingly, the process according to the present invention can be used in the work-up of any process for the preparation of Saflufenacil, i.e. Compound (I), where Compound (I) is obtained in solubilized form in the organic solvent phase.

Furthermore. the process according to the present invention can be used to separate Compound (I), which is present in solubilized form in an organic solvent phase, from impurities.

Solvents suitable for these reactions are, depending on the temperature range of the final reaction step, aliphatic, cycloaliphatic or aromatic hydrocarbons, such as pentane, hexane, cyclopentane, cyclohexane, toluene, xylene, chlorinated aliphatic and aromatic hydrocarbons, such as dichloromethane, trichloromethane, 1,2-dichloroethane, 1,1,2,2-tetrachloroethane, chlorobenzene, 1,2-, 1,3- or 1,4-dichlorobenzene, chlorotoluenes, dichlorotoluenes, open-chain dialkyl ethers, such as diethyl ether, di-n-propyl ether, diisopropyl ether, methyl tert-butyl ether, cyclic ethers, such as tetrahydrofuran, 1,4-dioxane, anisole, glycol ethers, such as dimethyl glycol ether, diethyl glycol ether, diethylene glycol dimethyl ether, diethylene glycol diethyl ether, C₁-C₄-alcohols, such as methanol, ethanol, n-propanol, isopropanol, n-butanol, aliphatic C₁-C₆-alkyl carboxylates, such as methyl acetate, ethyl acetate or n-butyl acetate; ketones, such as acetone, methyl ethyl ketone, methyl isopropyl ketone, methyl isobutyl ketone, butanone, carbonates, such as diethyl carbonate and ethylene carbonate, N, Ndialkylamides, such as N,N-dimethylformamide or N, N-dimethylacetamide, N-alkyllactams, such as N-methylpyrrolidone, sulfoxides, such as dimethyl sulfoxide, tetraalkyl ureas, such as tetramethyl urea, tetraethyl urea, tetrabutyl ureas, dimethylethylene urea, dimethylpropylene urea or mixtures of these solvents.

Preferred solvents are N, N-dimethylformamide, N-methylpyrrolidone, acetone, dichloromethane, tetrahydrofuran (THF), toluene, chlorobenzene, methyl acetate, ethyl acetate, butyl acetate or mixtures of these solvents.

In one embodiment, Saflufenacil is obtained by alkylating Compound (II) as described in WO2006/125746:

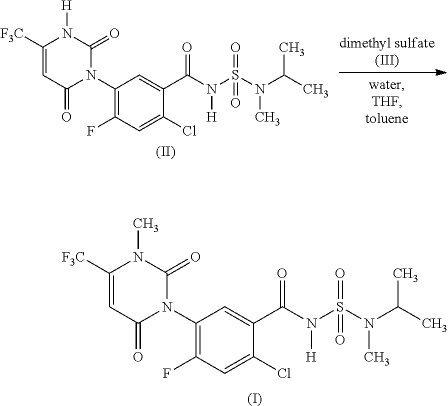

Work-up of the reaction mixture to obtain Saflufenacil can be carried out by methods customary for this purpose. In general, the solvent used is removed by suitable processes, for example by distillation. Saflufenacil can then be taken up in a water-immiscible organic solvent, any impurities are then extracted using water which, if appropriate, is acidified. Upon phase separation, the organic phase comprising the product is dried, and the solvent is removed under reduced pressure. For further purification, it is possible to use usual processes such as crystallization, precipitation or chromatography.

When Compound (I) is crystallized from the organic solvent phase, wetcake can be obtained by centrifugation. The mother liquor comprises residues of Compound (I) and unreacted Compound (II) as well as several organic impurities.

The mother liquor comprises the organic solvents used in the final reaction step for preparing Compound (I), e.g. N,N-dimethylformamide, N-methylpyrrolidone, acetone, dichloromethane, tetrahydrofuran (THF), toluene, chlorobenzene, methyl acetate, ethyl acetate, butyl acetate or mixtures of these solvents, preferably, a mixture of tetrahydrofuran (THF) and toluene.

The concentration of the organic solvents in the mother liquor is >80% [w/w], preferably >90% [w/w].

The water content of the mother liquor is generally <1% [w/w], preferably <0.5% [w/w], more preferably <0.2% [w/w].

The concentration of residual Compound (I) in the mother liquor is generally between 1-8% [w/w], often between 2-5% [w/w].

In addition to Compound (I), the mother liquor comprises unreacted Compound (II) and organic impurities, e.g. compounds (IV) and (V):

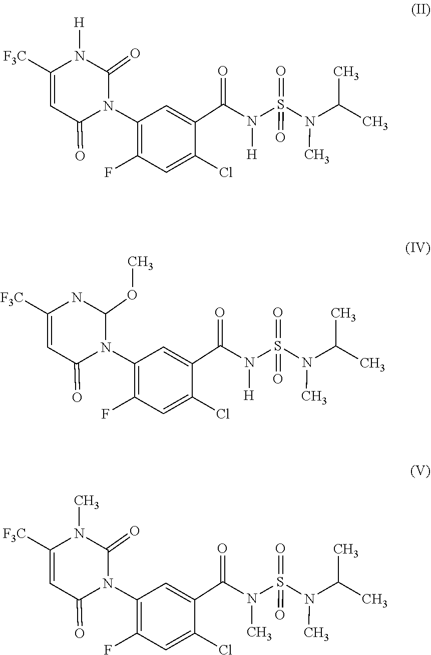 [SMILES: CC(C)N(C)S(=O)(=O)NC(=O)c1cc(-n2c(=O)cc(C(F)(F)F)[nH]c2=O)c(F)cc1Cl ; COC1NC(C(F)(F)F)=CC(=O)N1c1cc(C(=O)NS(=O)(=O)N(C)C(C)C)c(Cl)cc1F ; CC(C)N(C)S(=O)(=O)N(C)C(=O)c1cc(-n2c(=O)cc(C(F)(F)F)n(C)c2=O)c(F)cc1Cl]

Furthermore, Compound (I) is susceptible to rapid degradation to compound (VI) with time and elevated pH (>7):

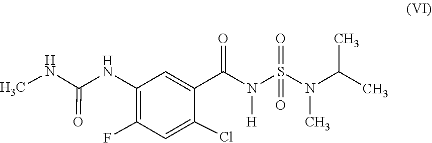 [SMILES: CNC(=O)Nc1cc(C(=O)NS(=O)(=O)N(C)C(C)C)c(Cl)cc1F]

The rapid degradation of Compound (I) to Compound (VI) poses a significant challenge for developing a process to recover Compound (I) from the organic mother liquor with high yield before significant amounts of the compound are lost by degradation. Furthermore, the process should provide Compound (I) with high purity, i.e. without the presence of significant amounts of organic impurities.

The problem of recovering Compound (I) from the organic mother liquor without significant degradation and without significant amounts of unwanted impurities has been achieved by the following process (see exemplary Scheme 1):
STEP I: Extraction of Compound (I) from the organic mother liquor (2) into an aqueous phase (aqueous process stream (3))
STEP II: Back-extraction of Compound (I) from the aqueous process stream (3) into an organic phase (9)

### STEP I: Extraction of Compound (I) From the Organic Mother Liquor (2) Into an Aqueous Phase (Aqueous Process Stream (3))

### I.a) Preparation of an Aqueous Basic Buffer Solution (1) for Conversion of Compound (I) Into a Salt

For extraction of Compound (I) from the organic mother liquor into an aqueous phase, the compound is converted into a salt, which readily dissolves in the aqueous phase. This can be achieved by shifting the pH to a basic value by adding a suitable base. Accordingly, the organic mother liquor stream (2) is mixed with a base, preferably an aqueous basic buffer solution (1).

Suitable bases for adjusting the pH to convert Compound (I) into a salt are all customary organic and inorganic bases.

Preferred bases are selected from the group consisting of alkali metal and alkaline earth metal hydroxides, such as sodium hydroxide, potassium hydroxide and lithium hydroxide, alkali metal and alkaline earth metal oxides, such as calcium oxide, alkali metal and alkaline earth metal carbonates, such as lithium carbonate, sodium carbonate, potassium carbonate, cesium carbonate, magnesium carbonate, calcium carbonate, zinc carbonate, alkali metal bicarbonates, such as sodium bicarbonate and also ammonia or tertiary amines, such as triethylamine. Particularly preferred bases for adjusting the pH are selected from the group consisting of alkali metal and alkaline earth metal hydroxides, alkali metal and alkaline earth metal carbonates ammonia and also tertiary amines.

Most preferred bases for adjusting the pH to convert Compound (I) into a salt are aqueous basic buffer solutions of Na₂CO₃ or NH₄OH (0.1 to 1% [w/w]).

To prevent formation of an emulsion in the organic phase, the addition of a “density enhancer” to the aqueous basic buffer solution is recommendable. Suitable density enhancers are water soluble inorganic salts, for example sodium salts such as Na₂CO₃, NaCl, and Na₂SO₄, or ammonium salts such as (NH₄)₂SO₄. In a preferred embodiment, Na₂SO₄ is used as density enhancer.

In one embodiment, the solid density enhancer salts are dissolved in water. In another embodiment, a solution containing the density enhancer salt is prepared by blending an inorganic acid solution with the respective base solution, e.g. aqueous solutions of sulfuric acid and sodium hydroxide or ammonia.

The concentration of the density enhancer in the aqueous basic buffer solution (1) should be less than 5% [w/w], preferably between 1 and 3% [w/w].

The final aqueous basic buffer solution (1) for extraction comprises the aqueous basic buffer solution and, optionally, the density enhancer. In one embodiment, the aqueous basic buffer solution is charged to the solution of the density enhancer.

The pH of the aqueous basic buffer solution (1) should be higher than 8, preferably higher than 9, more preferably higher than 10, and most preferably 11.

### I.b) Extraction of the Salt of Compound (I) Into the Aqueous Process Stream (3)

Extraction of the salt of Compound (I) from the mother liquor into the aqueous process stream is achieved by mixing the mother liquor (2) with the aqueous basic buffer solution (1) and adequate agitation. The extraction relies on basic pH control ranging from 7-10, preferably from 7.5 to 9, more preferably from 7.8-8.5, to extract almost all Compound (I) from the mother liquor as a salt into an aqueous phase.

The weight ratio of the aqueous phase to the organic phase for the extraction (Aq:Org ratio) is between 0.15:1.0 and 1.0:1.0, preferably between 0.6:1 and 0.9:1.

Compound (I) starts to decompose in the presence of base on a timescale of minutes. After one hour, Compound (I) is degraded up to 100% to Compound (VI) at pH 11. For this reason, the extraction residence time should be as low as possible and Compound (I) extracted to the aqueous phase is back-extracted by decreasing the pH in the presence of organic solvent (see STEP II below).

Mixing and separation of the two phases are conveniently done at ambient temperature and pressure. It is advantageous to pre-cool both the organic mother liquor (2) and the aqueous basic buffer solution (1) to allow extraction at temperatures between 10 and 60° C. Subsequently, the two phases, organic layer (4) and aqueous layer (3), are separated.

To recover most of the salt of Compound (I) in the aqueous process stream (3), the extraction should be carried out with at least two stages, preferably with three stages, more preferably with four stages.

Extraction is commonly achieved by shaking the mother liquor (2) and the aqueous basic buffer solution (1) in a suitable vessel, e.g. in a separating funnel. The extraction can be performed batchwise, or alternatively as a continuous liquid-liquid extraction.

Extraction and separation should be achieved within a short period of time, so that the extracted aqueous phase comprising Compound (I) can be back-extracted by decreasing the pH within 30 min, preferably within 15 min, more preferably, within 5 min.

It is possible to perform the extraction of Compound (I) from the mother liquor to the aqueous phase in a multistage extractor/separator, e.g. Robatel Model LX-204, which allows for very low residence time, i.e. less than 30 min, preferably less than 15 min, more preferably less than 5 min. The aqueous phase (aqueous basic buffer solution (1)) is fed into the top of the extractor and the organic phase (mother liquor (2)) is fed into the bottom. Under centrifugal force, the streams mix and separate in each phase of the extractor. The organic phase (4) moves up to the top discharge port and the aqueous phase (aqueous process stream (3)) moves down to the bottom discharge port.

The target recovery for Compound (I) from the organic mother liquor (2) is more than 70%, preferably more than 80%, most preferably more than 90%.

### STEP II: Back-Extraction of Compound (I) From the Aqueous Process Stream (3) Into an Organic Phase (9)

The aqueous process stream (3) obtained in STEP Ib) can be acidified with a suitable acid, converting the salt into Compound (I). As Compound (I) is not soluble in water, it will crystallize from the aqueous stream. Once acidified, Compound (I) is stable and can be kept for further use. Compound (I) can be recovered by filtration of the aqueous phase or can be re-solubilized by adding a suitable organic solvent (as above), the resulting solution can be further processed as described below.

Generally, the aqueous phase obtained in STEP Ib) is acidified without delay, i.e. Compound (I) extracted to the aqueous process stream (3) is back-extracted by decreasing the pH to below 6, preferably to below 4, more preferably to 1.9 and 2.5, most preferably to 2, in the presence of organic solvent, preferably consistent with the native mother liquor (2). The pH during back-extraction is adjusted by adding the suitable acid, so that the pH of the aqueous process stream is maintained at the desired pH.

Acidification for the back-extraction is achieved by adding a suitable acid. Suitable acids are commonly used organic and inorganic acids. Examples of inorganic acids are hydrohalic acids and oxygen acids, especially hydrochloric acid, sulfuric acid, nitric acid, phosphoric acid, phosphonic acid and phosphinic acid. Examples of organic acids are aliphatic and aromatic acids such as alkylsulfonic acids, arylsulfonic acids, mono-C₁-C₆-alkyl phosphates, di-C₁-C₅-alkyl phosphates, monoaryl phosphates, diaryl phosphates, alkylcarboxylic acids, haloalkylcarboxylic acids and heterocyclylcarboxylic acids, especially methanesulfonic acid, p-toluenesulfonic acid, citric acid, trifluoroacetic acid, acetic acid and proline. Preferred acids are hydrochloric acid, hydrobromic acid, sulfuric acid, nitric acid, and acetic acid, particularly preferred is sulfuric acid.

Solvent systems used for the back-extraction are water-immiscible, and Compound (I) has acceptable solubility therein. Suitable components of these solvent systems are aliphatic, cycloaliphatic or aromatic hydrocarbons, such as pentane, hexane, cyclopentane, cyclohexane, toluene, xylene, chlorinated aliphatic and aromatic hydrocarbons, such as dichloromethane, trichloromethane, 1,2-dichloroethane, 1,1,2,2-tetrachloroethane, chlorobenzene, 1,2-, 1,3- or 1,4-dichlorobenzene, chlorotoluenes, dichlorotoluenes, open-chain dialkyl ethers, such as diethyl ether, di-n-propyl ether, diisopropyl ether, methyl tert-butyl ether, cyclic ethers, such as tetrahydrofuran, 1,4-dioxane, anisole, glycol ethers, such as dimethyl glycol ether, diethyl glycol ether, diethylene glycol dimethyl ether, diethylene glycol diethyl ether, C₁-C₄-alcohols, such as methanol, ethanol, n-propanol, isopropanol, n-butanol, aliphatic C₁-C₆-alkyl carboxylates, such as methyl acetate, ethyl acetate or n-butyl acetate; ketones, such as acetone, methyl ethyl ketone, methyl isopropyl ketone, methyl isobutyl ketone, butanone, carbonates, such as diethyl carbonate and ethylene carbonate, N, Ndialkylamides, such as N, N-dimethylformamide or N,N-dimethylacetamide, N-alkyllactams, such as N-methylpyrrolidone, sulfoxides, such as dimethyl sulfoxide, tetraalkyl ureas, such as tetramethyl urea, tetraethyl urea, tetrabutyl ureas, dimethylethylene urea, dimethylpropylene urea or mixtures of these solvents.

Preferred components of these solvent systems are N, N-dimethylformamide, N-methylpyrrolidone, acetone, dichloromethane, tetrahydrofuran (THF), toluene, chlorobenzene, methyl acetate, ethyl acetate, butyl acetate or mixtures of these components. Particularly preferred for the back-extraction is a mixture of toluene and THF, e.g. in a ratio of 80:20 [v/v], preferably in a ratio of 90:10 [v/v] or in a ratio of 95:5 [v/v].

Back-extraction is commonly achieved by mixing the aqueous process stream (3) form STEP Ib) with the solvent system (5) in a suitable vessel, e.g. in a separating funnel, with adequate agitation and at the desired pH. The desired pH is adjusted by continuously dosing the suitable acid (6). The weight ratio of the aqueous process stream (3) to the organic phase (5) for the back-extraction (Aq:Org ratio) is between 0.5:1 to 1:0.5, preferably about 1:1.

The back-extraction can be performed batchwise, or alternatively as a continuous liquid-liquid extraction. Preferably, the back-extraction is performed continuously. Residence time for back-extraction can vary in a wide range, as Compound (I) is stable under these conditions. Practically, the approximate residence time in the back-extraction vessel varies between 20-40 min. Residence time can be 40-80 minutes total, if back-extraction is conducted in two consecutive vessels. The two-phase mixture is then sent to a decanter, where the phases are separated. The organic phase (9) comprises recovered Compounds (I) and (II) and significantly less organic impurities than the mother liquor.

The target recovery for Compound (I) from the aqueous process stream of STEP Ib) is more than 90%, preferably more than 95%, most preferably more than 99%.

The organic phase (9) obtained in STEP II), comprising purified Compound (I) and unreacted Compound (II), can be combined with the reaction mixture in the final step of the process for the preparation of compound (I) or can be recycled to workup after the final step in the process for the preparation of compound (I). By recycling this organic phase back to the process for the preparation of compound (I), the extraction allows for higher overall yield of the process, lower cost of goods, better solvent recovery, and a reduction in waste generation.

The overall target recovery for Compound (I) is from 75%-100% within the mother liquor stream, resulting in a yield improvement of 7-10% for Compound (I).

An example for a suitable set-up to perform STEPS I and II is depicted in scheme 1.

### Example 1

STEPS I) and II) were conducted at ambient temperature and pressure. The mother liquor comprised THF/Toluene as solvent system; Na₂SO₄ was used as density enhancer.

In STEP I, extraction was carried out in a Centrifugal Extractor (Robatel Model LX-204) with an Aq:Org ratio [w/w] of 0.75:1, the pH being adjusted to 8.1.

In STEP II, back-extraction was carried out as continuous liquid-liquid extraction, with an Aq:Org ratio [w/w] of 1:1, the pH being adjusted to 1.95,

TABLE 1
The data provided is the concentration [% w/w] of the respective
components in the process streams as shown in Scheme 1:

| | 2 | 3 | 4 | 7 | 8 | 9 |
|---|---|---|---|---|---|---|
| Cpd. (I) | 3.48 | 3.94 | 0.32 | 1.92 | 1.92 | 3.77 |
| Cpd. (II) | 0.10 | 0.13 | n.d. | 0.06 | 0.06 | 0.12 |
| Cpd. (IV) | 0.61 | 0.12 | 0.54 | 0.06 | 0.06 | 0.11 |
| Cpd. (V) | 0.66 | 0.00 | 0.69 | 0.00 | 0.00 | 0.00 |
| Cpd: (VI) | — | 0.05 | n.d. | 0.02 | 0.02 | 0.05 |

Compounds (I) and (II) are recovered from the mother liquor (2) in high yield in the organic stream (9) and can be recycled to the process for the production of Compound (I). Impurities (IV) and (V) are removed from the process stream. In the inventive process, degradation of Compound (I) to Compound (VI) is prevented.

### Example 2

In analogy to Example 1, the process was implemented into the production process for Compound (I). The data sets A-J show the results for independent campaigns, [% w/w] of the respective compounds in the relevant process streams (2, 4 and 9):

TABLE 2

| | | Cpd. | Cpd. | Cpd. | Cpd. | Cpd. |
|---|---|---|---|---|---|---|
| | | (I) | (II) | (IV) | (V) | (VI) |
| A | | | | | | |
| 2 | Mother Liquor Feed | 3.07 | 0.09 | 0.49 | 0.51 | 0.03 |
| 4 | Extractor Discharge Organic | 0.41 | 0.01 | 0.48 | 0.59 | 0.00 |
| B | | | | | | |
| 2 | Mother Liquor Feed | 3.47 | 0.13 | 0.49 | 0.48 | 0.07 |
| 4 | Extractor Discharge Organic | 0.28 | 0.01 | 0.45 | 0.56 | 0.00 |
| 9 | Recovered back-extractant | 2.97 | 0.14 | 0.20 | 0.03 | 0.03 |
| C | | | | | | |
| 2 | Mother Liquor Feed | 2.96 | 0.12 | 0.47 | 0.39 | 0.03 |
| 4 | Extractor Discharge Organic | 0.05 | 0.01 | 0.33 | 0.43 | 0.00 |
| 9 | Recovered back-extractant | 2.90 | 0.12 | 0.17 | 0.01 | 0.03 |
| D | | | | | | |
| 2 | Mother Liquor Feed | 2.79 | 0.15 | 0.45 | 0.36 | 0.04 |
| 4 | Extractor Discharge Organic | 0.04 | 0.01 | 0.34 | 0.42 | 0.00 |
| 9 | Recovered back-extractant | 2.84 | 0.15 | 0.15 | 0.00 | 0.04 |
| E | | | | | | |
| 2 | Mother Liquor Feed | 2.70 | 0.13 | 0.41 | 0.39 | 0.03 |
| 4 | Extractor Discharge Organic | 0.16 | 0.01 | 0.36 | 0.46 | 0.00 |
| 9 | Recovered back-extractant | 2.54 | 0.12 | 0.11 | 0.02 | 0.03 |
| F | | | | | | |
| 2 | Mother Liquor Feed | 3.03 | 0.17 | 0.42 | 0.40 | 0.03 |
| 4 | Extractor Discharge Organic | 0.20 | 0.01 | 0.39 | 0.46 | 0.00 |
| 9 | Recovered back-extractant | 2.88 | 0.17 | 0.12 | 0.03 | 0.03 |
| G | | | | | | |
| 2 | Mother Liquor Feed | 2.92 | 0.20 | 0.40 | 0.34 | 0.02 |
| 4 | Extractor Discharge Organic | 0.18 | 0.01 | 0.34 | 0.37 | 0.00 |
| 9 | Recovered back-extractant | 2.95 | 0.18 | 0.12 | 0.02 | 0.02 |
| H | | | | | | |
| 2 | Mother Liquor Feed | 2.88 | 0.18 | 0.43 | 0.33 | 0.02 |
| 4 | Extractor Discharge Organic | 0.19 | 0.01 | 0.34 | 0.36 | 0.00 |
| 9 | Recovered back-extractant | 2.60 | 0.16 | 0.12 | 0.03 | 0.02 |
| J | | | | | | |
| 2 | Mother Liquor Feed | 3.02 | 0.19 | 0.51 | 0.42 | 0.04 |
| 4 | Extractor Discharge Organic | 0.12 | 0.00 | 0.41 | 0.44 | 0.00 |
| 9 | Recovered back-extractant | 3.05 | 0.19 | 0.15 | 0.04 | 0.04 |

1. A process for increasing a yield of a process for preparation of Compound (I), in which Compound (I) is obtained in solubilized form in an organic solvent phase, crystallized, and separated from the mother liquor, comprising 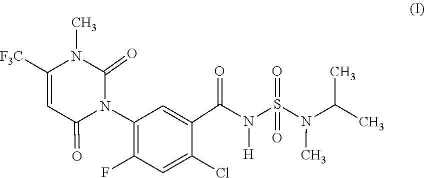 [SMILES: CC(C)N(C)S(=O)(=O)NC(=O)c1cc(-n2c(=O)cc(C(F)(F)F)n(C)c2=O)c(F)cc1Cl]
STEP I) wherein the mother liquor is extracted with a basic buffer solution, adjusting the pH between 7 and 10, and extracting Compound (I) into the aqueous phase; and
STEP II) wherein Compound (I) is back-extracted from the aqueous phase by decreasing the pH to below 6 in the presence of the organic solvent.

2. The process as claimed in claim 1, wherein the organic solvent comprising extracted Compound (I) obtained in STEP (II) is recycled into the process for the preparation of Compound (I).

3. The process as claimed in claim 1, wherein the pH of the basic buffer solution is between 7.5 and 9.

4. The process as claimed in claim 1, wherein the basic buffer solution comprises a base selected from the group consisting of alkali metal and alkaline earth metal hydroxides, alkali metal and alkaline earth metal oxides, alkali metal and alkaline earth metal carbonates, alkali metal bicarbonates, ammonia, and tertiary amines.

5. The process as claimed claim 1, wherein a weight ratio of the basic buffer solution and the mother liquor and is between 0.15:1.0 and 1.0:1.0.

6. The process as claimed in claim 1, wherein the extraction time of STEP I) is below 30 min.

7. A process for recovering a Compound (I) from a mother liquor comprising more than 80% of an organic solvent, Compound (I), and optionally Compounds (II), (IV), or (V), 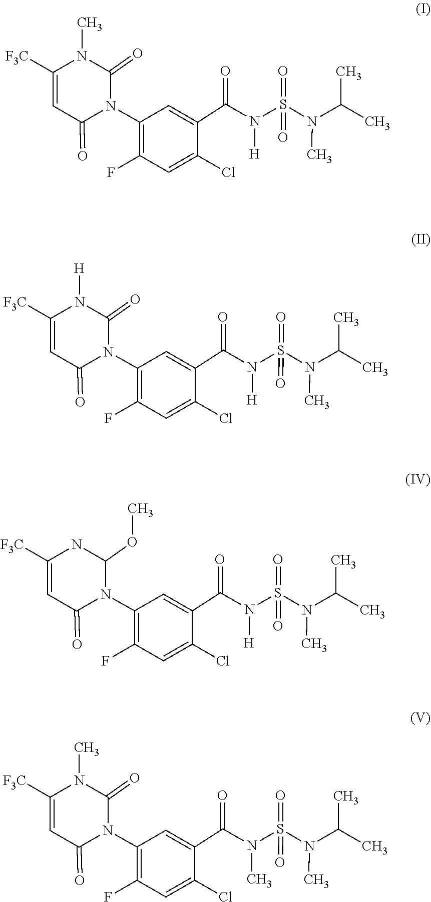 [SMILES: CC(C)N(C)S(=O)(=O)NC(=O)c1cc(-n2c(=O)cc(C(F)(F)F)n(C)c2=O)c(F)cc1Cl ; CC(C)N(C)S(=O)(=O)NC(=O)c1cc(-n2c(=O)cc(C(F)(F)F)[nH]c2=O)c(F)cc1Cl ; COC1NC(C(F)(F)F)=CC(=O)N1c1cc(C(=O)NS(=O)(=O)N(C)C(C)C)c(Cl)cc1F ; CC(C)N(C)S(=O)(=O)N(C)C(=O)c1cc(-n2c(=O)cc(C(F)(F)F)n(C)c2=O)c(F)cc1Cl]
wherein
STEP I) the mother liquor is extracted with a basic buffer solution, adjusting the pH between 7 and 10, and extracting Compound (I) into the aqueous phase; and
STEP II) Compound (I) is back-extracted from the aqueous phase by decreasing the pH to below 6 in the presence of the organic solvent.

8. The process as claimed in claim 7, wherein the pH of the basic buffer solution is between 7.5 and 9.

9. The process as claimed in claim 8, wherein the basic buffer solution comprises a base selected from the group consisting of alkali metal and alkaline earth metal hydroxides, alkali metal and alkaline earth metal oxides, alkali metal and alkaline earth metal carbonates, alkali metal bicarbonates, ammonia, and tertiary amines.

10. The process as claimed in claim 7, wherein a weight ratio of the basic buffer solution and the mother liquor and is between 0.15:1.0 and 1.0:1.0.

11. The process as claimed in claim 7, wherein the extraction time of STEP I) is below 30 min.

12. The process as claimed in claim 7, wherein the organic phase obtained in STEP II comprises more than 90% of the Compound (I) comprised in the organic mother liquor of STEP I.

13. The process as claimed in claim 7, wherein the organic phase obtained in STEP II comprises less than 50% of the Compound (IV) comprised in the organic mother liquor of STEP I.

14. The process as claimed in claim 7, wherein the organic phase obtained in STEP II comprises less than 80% of the Compound (V) comprised in the organic mother liquor of STEP I.

15. The process as claimed in claim 4 wherein the buffer solution comprises a base selected from the group consisting of sodium hydroxide, potassium hydroxide, lithium hydroxide, calcium oxide, lithium carbonate, sodium carbonate, potassium carbonate, cesium carbonate, magnesium carbonate, calcium carbonate, zinc carbonate, sodium bicarbonate, and triethylamine.
